(19) United States
(12) Patent Application Publication
(10) Pub. No.: US 2024/0058481 A1
(43) Pub. Date: Feb. 22, 2024

(54) RADIOLABELLED COMPOUNDS FOR DIAGNOSING CHOLINERGIC NEURODEGENERATIVE DISEASES

(71) Applicants: CENTRE NATIONAL DE LA RECHERCHE SCIENTIFIQUE, Paris (FR); INSTITUT NATIONAL DE LA SANTE ET DE LA RECHERCHE MEDICALE, Paris (FR); UNIVERSITÉ DE TOURS, Tours (FR); UNIVERSITE D'ORLEANS, Orleans (FR)

(72) Inventors: Sylvain ROUTIER, Tigy (FR); Frédéric BURON, Orleans (FR); Franck SUZENET, Saint Jean Le Blanc (FR); Nuno RODRIGUES, Mende (FR); Sylvie CHALON, Saint Cyr Sur Loire (FR); Johnny VERCOUILLIE, Amboise (FR); Denis GUILLOTEAU, Saint Cyr Sur Loire (FR); Patrick EMOND, Vouvray (FR); Hamid MARZAG, Orleans (FR)

(21) Appl. No.: 18/253,953

(22) PCT Filed: Nov. 23, 2021
(86) PCT No.: PCT/EP2021/082619

§ 371 Date: May 23, 2023

(30) Foreign Application Priority Data
Nov. 23, 2020 (FR) FR2012024

(51) Int. Cl. A61K 51/04; C07D 211/14; C07F 5/02

(52) U.S. Cl. CPC A61K 51/0455; C07D 211/14; C07F 5/025

(57) ABSTRACT

The present invention relates to the radiolabelled compound of enantiomer (R,R) of 5-fluoro-3-4(-phenylpiperidin-1-yl)-1,2,3,4-tetrahydronaphthalen-2-ol, and to this compound for use thereof in an in vivo diagnosis method, in particular of a cholinergic neurodegenerative disease selected for example from the group formed by Alzheimer's disease, dysmnesia, learning disability, schizophrenia, cognitive dysfunction, hyperactivity disorder, anxiety neurosis, depression, analgesia and Parkinson's disease.

The subject of the present invention concerns novel compounds radiolabelled with fluorine-18, the method of preparation and uses thereof, in particular for the diagnosis of cholinergic neurodegenerative diseases.

Cholinergic systems are involved in multiple functions, and much information has been gathered in recent years on the mechanisms of the synthesis, storage, degradation and effects of acetylcholine on the numerous sub-types of muscarinic and cholinergic receptors. Within this context, the vesicular acetylcholine transporter (VAChT) plays a major role which means that, together with the location thereof in the endings of the cholinergic neurons, it is a target of choice for studying the functioning and integrity of cholinergic systems. At central level, these systems are particularly involved in cognitive processes, and in vivo investigation of VAChT is of interest both for the diagnosis and for the follow-up of neurodegenerative disorders associated with alterations of these processes. This is case for Alzheimer's disease (AD) which is the most frequent cause of dementia and currently affects 900 000 people in France, amounting to an issue of public health that is increasingly acute on account of ageing of the population. In addition, dysfunction of the cholinergic systems translating as changes in the expression of VAChT have been described for other neurodegenerative disorders such as in Parkinsonian dementia (Kotagal et al. Neurosci Lett. 2012 Apr 18;514(2):169-72. doi: 10.1016/j. neulet.2012.02.083, Kuhl et al Ann Neurol. 1996 Sep;40(3):399-410. doi: 10.1002/ana.410400309), progressive supranuclear palsy (Mazère et al. Radiology. 2012 Nov;265(2):537-43. doi: 10.1148/radiol.12112650), multiple system atrophy (Mazère et al. Neuroimage Clin. 2013 Aug 8;3:212-7. doi: 10.1016/j.nicl.2013.07.012), Lewy body dementia (Mazère et al. J Nucl Med. 2017 Jan;58(1):123-128. doi: 10.2967/jnumed. 116.176180; Nejad-Davarani et al. Mol Psychiatry. 2019 Mar;24(3):322-327. doi: 10.1038/s41380-018-0130-5), or in peripheral disorders such as cancer (Stokholm et al. Eur J Nucl Med Mol Imaging. 2016 May;43(5):906-910. doi: 10.1007/s00259-015-3143-1), or heart disorders (Durand et al. Am J Physiol Heart Circ Physiol. 2015 Aug 15;309(4): H655-62. doi: 10.1152/ajpheart.00114.2015).

The most relevant method for in vivo investigation of VAChT is positron emission tomography (PET) which requires the use of tracers emitting beta+ radiation, in particular fluorine-18 (¹⁸F), specific to the molecular target of interest.

Investigation of VAChT is of interest both for the diagnosis and for the follow-up of the aforementioned neurodegenerative diseases. It can be considered to be complementary to the imaging of abnormal proteins characteristic of these diseases. For example, the presence of beta-amyloid plaques detected by imaging is not always correlated with the intensity of clinical scores during the course of Alzheimer's disease (e.g. Stephen et al. J Alzheimer Dis. 2017;59(2):695-705. doi: 10.3233/JAD-170092). On the other hand, alteration of the cholinergic systems is necessarily linked to neuropsychiatric symptoms (e.g. Sultzer et al. Brain. 2018 Mar 1;141(3):626-628. doi: 10.1093/brain/awy040). Within this context, a very recent study has just shown that in vivo investigation of VAChT, during the course of Alzheimer's disease, allows the evidencing of cholinergic denervation which is more sensitive and better correlated with cognitive symptoms than investigation of β-amyloid plaques or of cerebral metabolism (Aghourian et al. Mol Psychiatry. 2017 Nov;22(11):1531-1538. doi: 10.1038/mp.2017.183.).

Solely iodobenzovesamicol labelled with iodine-123 ([¹²³I]IBVM) has been qualified to date as imaging marker useful for quantification of VAChT density in the brain (Mazère et al. Neuroimage. 2008 Mar 1;40(1):280-8. doi: 10.1016/j.neuroimage.2007.11.028). However, this molecule has exhibited non-specific binding, in particular binding to the sigma receptors. In addition, this tracer labelled with iodine-123 can only be used in single-photon emission computed tomography (SPECT), a molecular imaging method less adapted to in vivo quantification than PET. These two points therefore restrict the use of IBVM as tracer for clinical use.

It is therefore the object of the present invention to provide a novel radiolabelled compound allowing the diagnosis of cholinergic neurodegenerative diseases.

A further object of the invention is to provide a compound for the diagnosis of cholinergic neurodegenerative diseases that is adapted for positron emission tomography (PET).

A further object of the invention is to provide a PET tracer that is efficient in vivo with very good passing of the blood-brain barrier, having specific accumulation in the cerebral regions where VAChT transporters are located, and having good in vivo stability.

Therefore, the present invention relates to the compound having the following formula (I):

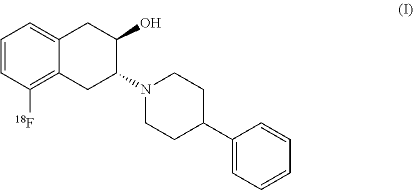 [SMILES: O[C@@H]1Cc2cccc([18F])c2C[C@H]1N1CCC(c2ccccc2)CC1]

The formula (I) compound of the invention is a radiolabelled compound and corresponds to the radiolabelled enantiomer (R,R) of 5-fluoro-3-4(-phenylpiperidin-1-yl)-1,2,3,4-tetrahydronaphthalen-2-ol (FBVM).

This compound is also called (−)-(R,R)-5-[¹⁸F]-FBVM.

The formula (I) compound of the invention is optically pure.

The present invention also relates to the formula (I) compound such as defined above, for use in an in vivo diagnosis method.

The present invention also concerns the formula (I) compound such as defined above, for use thereof in a method for in vivo diagnosis a cholinergic neurodegenerative disease.

In one embodiment, the cholinergic neurodegenerative disease is selected from the group formed by Alzheimer's disease, dysmnesia, learning disability, schizophrenia, cognitive dysfunction, hyperactivity disorder, anxiety neurosis, depression, analgesia and Parkinson's disease.

The invention therefore surmounts the barrier of early diagnosis i.e. before the onset of clinical signs of neurodegenerative disorders such as Alzheimer's disease by means of the quantitative in vivo imaging of VAChT, the decrease in the latter being an appreciable index of loss of cholinergic neurons which, on and after a certain threshold of loss of cholinergic neurons, is associated with the subsequent onset of cognitive disorders characteristic of the disease. This early detection is a major challenge since treatments, that are actively being sought, will be all the more efficient if they are administered at the first stages of the disease: the asymptomatic phases.

By comparison for example, 5(−)-[¹⁸F]FEOBV (5-fluoroethoxybenzovesamicol) has been described as a PET tracer targeting VAChT (Mulholland et al. 1998). However, the inventors have shown a better potential of the compound of the invention compared with this product which appears to be metabolised faster, leading to scattering of radioactivity. In addition, the accumulation in the brain of the compound of the invention is qualitatively similar it is true to that of FEOBV, but it is quantitatively greater.

On account of the arylfluorinated structure of the (−)-(R,R)-5-[¹⁸F]FBVM compound of the invention, it is not or only scarcely likely to lose its radioactive fluorine, contrary to 5-[¹⁸F]FEOBV which is radiolabelled at aliphatic position.

The molecule radiolabelled with optically pure fluorine-18 of the invention, namely the compound of formula (I), specifically binds to VAChT. As shown below, the present invention based on the structure of molecules of (benzo)vesamicol type, allows a good level of in vitro specificity to be reached, and the first radioligand has already exhibited its potential as in vivo PET tracer in the rat having regard to the excellent passing thereof through the blood-brain barrier, to the specific accumulation thereof in the cerebral regions where VAChT transporters are located, and to the good in vivo stability thereof.

The formula (I) compound of the invention can therefore also be used as tracer for PET quantification of VAChT.

The formula (I) compound of the invention can also be used as reagent for mapping of VAChT (radioactive indicator) or the like, which can be used for positron emission tomography (PET).

The compound of the invention can also be used to monitor and control the progression of the above-mentioned diseases, or to monitor the efficacy of the treatment of these diseases.

The present invention also relates to a method for preparing the compound of formula (I) such as defined above, comprising a step (a) to prepare a reaction mixture by adding a compound of following formula (III):

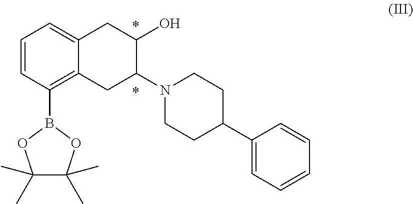 [SMILES: CC1(C)OB(c2cccc3c2CC(N2CCC(c4ccccc4)CC2)C(O)C3)OC1(C)C]

to reactive ¹⁸F fluorine ions,
followed by a step (b) for chiral separation of said reaction mixture obtained after step (a).

In one embodiment, step (a) is conducted in the presence of Cu(OTf)₂.

Preferably, step (a) is conducted in a mixture of DMF and pyridine as solvent.

In one embodiment, step (a) of the method of the invention is conducted in the presence of Cu(OTf)₂ in a mixture of DMF and pyridine.

In one embodiment, step (a) comprises a step to heat the reaction mixture to a temperature of from 90° C. to 120° C., preferably of 100° C.

In one embodiment, this heating is carried out for a time of 2 minutes to 30 minutes, preferably for a time of 10 minutes.

Preferably, the above-mentioned heating step is followed by a cooling step down to a temperature of 25° C. to 40° C., preferably of 30° C.

In one embodiment, step (a) of the method of the invention comprises a step to heat the reaction mixture to a temperature of 90° C. to 120° C., preferably of 100° C., for a time of 2 minutes to 30 minutes, preferably for a time of 10 minutes, said heating step preferably being followed by a cooling step down to a temperature of 25° C. to 40° C., preferably of 30° C.

In one embodiment, the chiral separation step (b) is performed by loading the reaction mixture obtained after step (a) onto a semi-preparative chiral column having a chiral stationary phase, in particular Chiral pak IA, Daicel 10*250 mm.

As mobile chiral phase, preferable use is made of a mobile phase comprising a mixture of acetonitrile, ammonium acetate and methanol.

Preferably, the mobile phase comprises from 50% to 90% by volume of acetonitrile, from 0% to 20% by volume of ammonium acetate and from 0% to 40% by volume of methanol, the percentages being calculated relative to the total volume of said mobile phase.

One preferred mobile phase of the invention comprises 70% by volume of acetonitrile, 10% by volume of ammonium acetate and 20% by volume of methanol, the percentages being calculated relative to the total volume of said mobile phase.

In one embodiment, the formula (III) compound is obtained by diazotization reaction of a compound of following formula (IV):

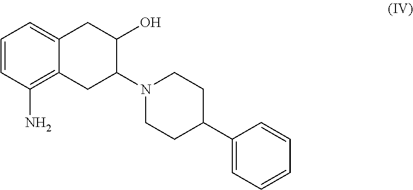 [SMILES: Nc1cccc2c1CC(N1CCC(c3ccccc3)CC1)C(O)C2]

followed by a substitution reaction by a halogen, such as iodine, on the diazo compound obtained after the above-mentioned diazotization reaction.
to obtain a compound of following formula (V):

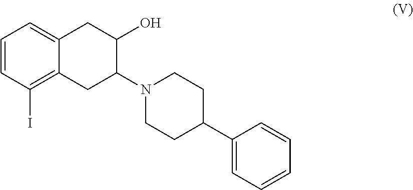 [SMILES: OC1Cc2cccc(I)c2CC1N1CCC(c2ccccc2)CC1]

and
   conversion of the compound of formula (V) by Miyaura borylation to obtain a compound of formula (III).

In one embodiment, the formula (III) compound is obtained with a method comprising the following steps:
the reaction of the compound of following formula (IV):

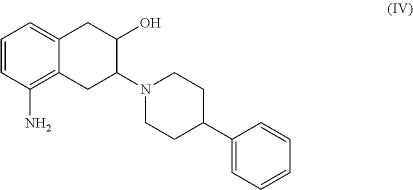 [SMILES: Nc1cccc2c1CC(N1CCC(c3ccccc3)CC1)C(O)C2]

with sodium nitrite, in particular in the presence of PTSA monohydrate, followed by the addition of potassium iodide,
to obtain a compound of following formula (V):

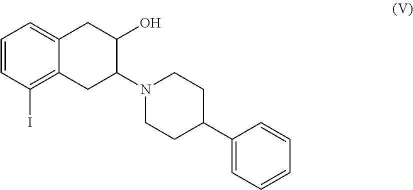 [SMILES: OC1Cc2cccc(I)c2CC1N1CCC(c2ccccc2)CC1]

and
   conversion of the formula (V) compound by Miyaura borylation to obtain a compound of formula (III).

The above-mentioned diazotization reaction step is conducted in a solvent suitable for this reaction. For example, mention can be made of toluene, dioxane, whether or not in the presence of ethanol and water.

The above-mentioned reaction step is preferably performed in acetonitrile, in particular at ambient temperature.

Preferably, this reaction step is conducted for 4 hours.

The conversion step of the formula (V) compound is preferably conducted in the presence of potassium acetate in solvents of amide type, for example DMA or DMF, and preferably DMF.

The above-mentioned reaction step is preferably conducted at a temperature of 80° C. to 110° C., preferably of 90° C., up until disappearance of the starting product, and in particular for 1 hour.

The present invention also relates to the compound of following formula (II):

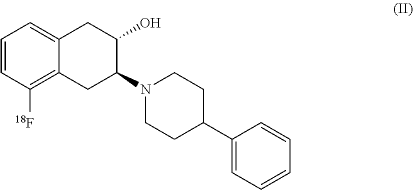 [SMILES: O[C@H]1Cc2cccc([18F])c2C[C@@H]1N1CCC(c2ccccc2)CC1]

The formula (II) compound of the invention is a radiolabelled compound and corresponds to the radiolabelled enantiomer (S,S) of 5-fluoro-3-4(-phenylpiperidin-1-yl)-1,2,3,4-tetrahydronaphthalen-2-ol (FBVM).

This compound is also called (+)-(S,S)-5-[¹⁸F]-FBVM.

This compound can be obtained with the method described above for the compound of formula (I).

The present invention also relates to the compound having the following formula (III):

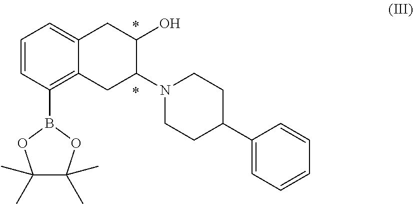 [SMILES: CC1(C)OB(c2cccc3c2CC(N2CCC(c4ccccc4)CC2)C(O)C3)OC1(C)C]

### EXAMPLES

### Synthesis of the Compounds rac (+/−)-5-FBVM trans, (R,R)-(−)-5-FBVM and (S,S)-(+)-5-FBVM and Determination of the Absolute Stereochemistry Thereof

The compound rac (+/−)-5-FBVM was synthesised following the synthesis route described in Schemes 1 and 2. 1-aminonaphthalene 1 was reduced to 5,8-dihydronaphthalene 2 using Birch conditions, after which the amine function was protected with the trifluoroacetic anhydride in the presence of triethylamine to arrive at compound 3 with a yield of 89%. This compound was used in an epoxidation reaction to give compound 4 with a yield of 85%. Opening of the epoxide in the presence of commercial 4-phenylpiperidine and triethylamine in the ethanol reflux, followed by deprotection of the amine function in a basic medium, led to the two regioisomers 5-R1 trans and 5-R2 trans with an overall yield of 60%. The two regioisomers 5-R1 trans and 5-R2 trans were separated and then characterized.

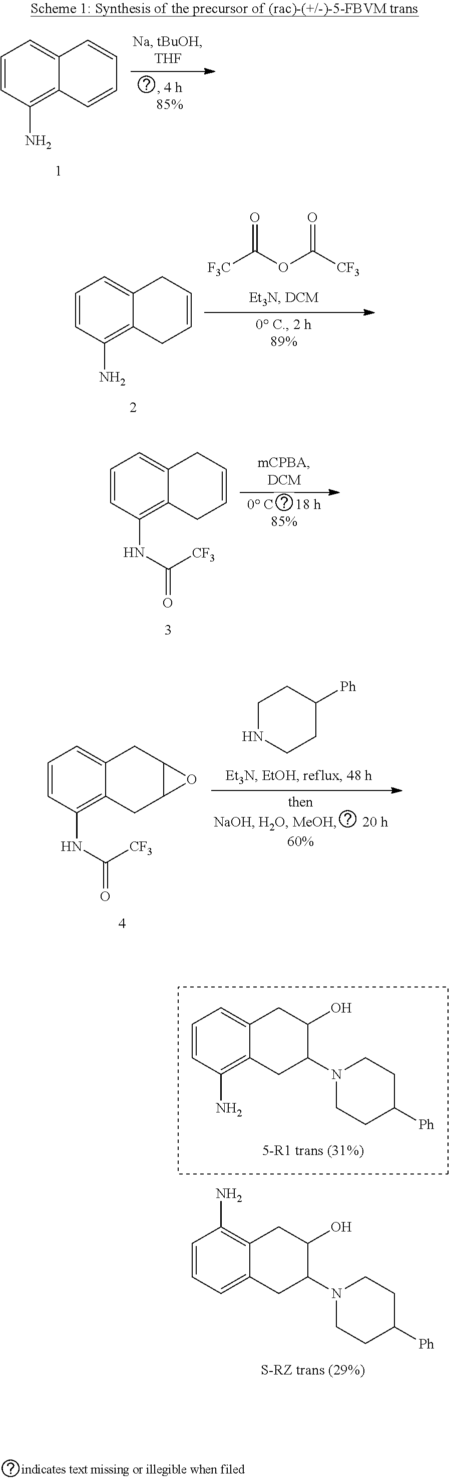

5.8-Dihydronaphthalen-1-amine (2)

In a round-bottom flask, compound 1 (16.0 g, 112 mmol) was solubilised in anhydrous THF (180 mL) under an argon atmosphere, and sodium 6.4 g, 279 mmol, 2.5 equivalents) was added portion-wise for 15 minutes. After agitation at ambient temperature for 10 min, a solution of tert-BuOH (26.8 ml, 279 mmol, 2.5 equiv.) in anhydrous THF (70 mL) was added dropwise for 30 min (exothermal reaction). The reaction was left under agitation at ambient temperature for 4 h. The mixture was filtered in vacuo to collect the sodium that had not reacted which was destroyed in isopropanol at 0° C. The filtrate was concentrated and dried under reduced pressure. The crude residue was dissolved in Et₂O (120 mL) at 0° C., after which water (120 mL) was slowly added. The mixture was transferred to a separation funnel, and the organic phase was separated. The aqueous phase was extracted with Et₂O (3×10 mL), and the organic phases were combined, dried over MgSO₄, filtered and concentrated under reduced pressure. The crude residue was purified by flash chromatography on a silica gel column (EP/AcOEt 95:5) to obtain the desired product 2 in the form of a dark brown syrup (13.8 g, 85%). Mp: 236-237° C.

IR: 3441, 3372, 3024, 2903, 2841, 2815, 2607, 2591, 1584, 1584, 1567, 1536, 1465, 1430, 1343, 1301, 1246, 1141, 1068, 1005, 769, 700, 668. ¹H NMR (400 MHZ, DMSO-d6) δ10.32 (brs, 2H, NH₂), 7.34 (d, J=7.7 Hz, 1H, H-Ar), 7.24 (t, J=7.7 Hz, 1H, H-Ar), 7.16 (d, J=7.7 Hz, 1H, H-Ar), 5.88 (s, 2H, 2=CH), 3.53-3.32 (m, 4H, 2 CH₂). ¹³C NMR (101 MHZ, DMSO-d6) δ135.6 (Cq), 130.3 (Cq), 128.2 (CH), 128.1 (Cq), 126.7 (CH), 124.0 (CH), 122.9 (CH), 120.9 (CH), 28.8 (CH₂), 24.6 (CH₂).

N-(5,8-Dihydronaphthalen-1-yl)-2,2,2-trifluoroacetamide (3)

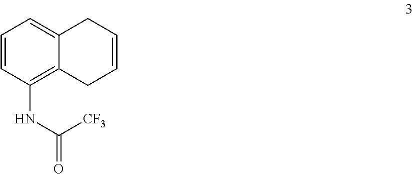 [SMILES: O=C(Nc1cccc2c1CC=CC2)C(F)(F)F]

In a round-bottom flask, compound 2 (13.0 g, 89.7 mmol) was solubilised in DCM (500 mL). The addition was made of Et₃N (25.0 ml, 179.4 mmol, 2.0 equivalents), and the reaction mixture was cooled to 0° C. Trifluoroacetic anhydride (17.6 ml, 134.6 mmol, 1.5 equiv.) was added dropwise and the reaction mixture was left under agitation at 0° C. for 2 h. The reaction was halted through a slow addition of water (250 mL). The mixture was transferred to a separation funnel, the organic layer was separated, washed in brine (100 mL) and then a saturated NaHCO₃ solution (100 mL), dried over MgSO₄, filtered and concentrated for drying under reduced pressure. The crude product was purified by flash chromatography on a silica gel column using EP/AcOEt (80:20) as eluent to obtain the desired product 3 in the form of a pink solid (19.3 g, 89%).

IR: 3277, 3034, 2930, 2900, 2815, 1703, 1610, 1586, 1550, 1467, 1347, 1291, 1256, 1185, 1151, 1068, 915, 782, 767, 700. ¹H NMR (250 MHz, CDCL₃) δ7.69 (brs, 1H, NH), 7.59 (dd, J=8.2, 1.3 Hz, 1H, H-Ar), 7.21 (d, J=7.9 Hz, 1H, H-Ar), 7.10-7.04 (m, 1H, H-Ar), 5.90 (ddddt, J=13.5, 12.0, 8.2, 3.4, 1.7 Hz, 2H, 2=CH), 3.49-3.38 (m, 2H, CH₂), 3.23 (tt, J=6.5, 2.4 Hz, 2H, CH₂). ¹³C NMR (63 MHZ, CDCL₃) δ155.2 (q, J=36.8 Hz, Cq), 135.7 (Cq), 132.1(Cq), 127.7(CH), 126.9 (Cq), 126.9(CH), 116.1 (q, J=288.9 Hz, CF₃), 29.8 (CH₂), 25.2 (CH₂). ¹⁹F NMR (235 MHZ, CDCL₃) δ-75.58 (d, J=1.3 Hz).

2,2,2-Trifluoro-N-(1a,2,7,7a-tetrahydronaphtho[2,3-b]oxiren-3-yl)acetamide (4)

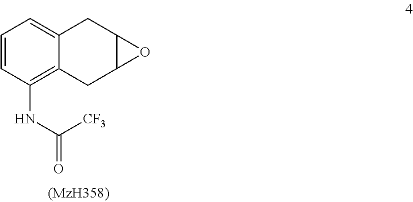 [SMILES: O=C(Nc1cccc2c1CC1OC1C2)C(F)(F)F]

In a round-bottom flask, compound 3 (10.0 g, 41.5 mmol) was solubilised in DCM (80 mL) and Et₂O (20 mL) under an argon atmosphere. The mixture was cooled to 0° C., after which mCPBA (8.585 g, 49.8 mmol, 1.2 equivalents) was added in a single addition. The reaction mixture was left under agitation at ambient temperature for 20h. The reaction was halted by adding a saturated aqueous solution of NaHCO₃ (60 mL). The organic and aqueous phases were separated and the aqueous phase was extracted with DCM (3×60 mL). The organic phases were then combined, dried over MgSO₄ and filtered. After removing the volatile substances under reduced pressure, the resulting crude product was solubilised in a minimum amount of DCM. The addition of Et₂O allowed the formation of a white solid which was collected by filtration, washed with Et₂O and dried in vacuo to obtain the desired epoxide 4 in the form of a white solid (8.4 g, 79%).

Mp: 102-103° C. IR: 3241, 3143, 3067, 3020, 2918, 2899, 2824, 1723, 1608, 1587, 1545, 1471, 1454, 1423, 1334, 1266, 1155, 1069, 998, 982, 922, 890, 860, 821, 787, 764, 741. ¹H NMR (400 MHZ, CDCL³) δ8.15 (brs, 1H, NH), 7.28-7.15 (m, 2H, 2 H-Ar), 7.05 (d, J=7.3 Hz, 1H, H-Ar), 3.57-3.49 (m, 2H, CH₂), 3.44-3.18 (m, 2H, CH₂), 2.93 (m, 2H, 2 CH). ¹³C NMR (101 MHZ, CDCL₃) δ155.4 (q, J=37.1 Hz, Cq), 133.2 (Cq), 132.4 (Cq), 129.2 (CH), 127.2 (CH), 126.8 (Cq), 123.6 (CH), 116.1 (q, J=288.9 Hz, CF₃), 52.3 (CH), 51.5 (CH), 30.1 (CH2), 25.1 (CH₂).

### Synthesis of Compounds 5-R1 and 5-R2:

In a round-bottom flask, the epoxide 4 (3.282 g, 12.8 mmol) was solubilised in EtOH (80 mL) under argon. 4-phenylpiperidine (2.059 g, 12.8 mmol, 1.0 equivalent) and triethylamine (3.558 ml, 25.5 mmol, 2.0 equivalents) were added and the reaction mixture was placed under reflux for 48 hours. The EtOH was removed under reduced pressure and the resulting crude product was solubilised in MeOH (70 mL). An aqueous solution of NaOH (6.128 g in 50 ml of water, 12.0 equivalents) was added and the reaction mixture was left under agitation at ambient temperature for 20 h. On completion of the reaction, a brown precipitate was formed. This precipitate was collected by filtration and solubilised in DCM for purification by flash chromatography on a silica gel column using DCM/MeOH (100:0 to 99.5:0.5, then 99:1 to 95:5) as mobile phase to obtain the desired products 5-R1 (1.249 g, 31%) and 5-R2 (1.189 g, 29%) as beige solids.

5-Amino-3-(4-phenylpiperidin-1-yl)-1,2,3,4-tetrahydronaphthalen-2-ol (5-R1):

Mp: 165-166° C. IR: 3453, 3372, 3061, 3023, 3005, 2929, 2903, 2846, 2846, 2814, 1618, 1586, 1494, 1466, 1439, 1409, 1374, 1331, 1300, 1250, 1141, 1068, 1005, 783, 765. HRMS (ESI⁺): calc. for C₂₁H₂₇N₂O [M+H]⁺: 323.21179 found: 323.21168. ¹H NMR (400 MHZ, CDCL₃) δ7.36-7.29 (m, 2H, H-Ar), 7.28-7.19 (m, 3H, 3H-Ar), 6.99 (t, J=7.7 Hz, 1H, H-Ar), 6.65-6.50 (m, 2H, 2 H-Ar), 4.19 (brs, 1H, OH), 3.87 (td, J=10.5, 5.5 Hz, 1H, CH), 3.60 (brs, 2H, NH₂), 3.25 (dd, J=15.9, 5.5 Hz, 1H, H of CH₂), 2.99 (dt, J=11.2, 3.4 Hz, 1H, H of CH₂), 2.93-2.76 (m, 4H, CH and H of 2 CH₂ and CH₂), 2.71 (dd, J=15.5, 5.6 Hz, 1H, H of CH₂), 2.62-2.40 (m, 3H, 2 H of 2 CH₂ and CH), 1.99-1.82 (m, 3H, CH₂ and 1H of CH₂), 1.76 (qd, J=12.4, 3.8 Hz, 1H, H of CH₂). ¹³C NMR (101 MHZ, CDCL₃) δ146.2 (Cq), 144.6 (Cq), 135.1 (Cq), 128.6 (2CH), 127.1 (CH), 127.0 (2CH), 126.4 (CH), 119.9 (CH), 119.9 (Cq), 112.8 (CH), 66.8 (CH), 65.4 (CH), 53.69 (CH₂), 45.1 (CH₂), 43.1 (CH), 38.3 (CH₂), 34.5 (CH₂), 34.0 (CH₂), 21.0 (CH₂). ¹⁹F NMR (376 MHZ, CDCL₃) δ-75.3.

8-Amino-3-(4-phenylpiperidin-1-yl)-1,2,3,4-tetrahydronaphthalen-2-ol (5-R2):

Mp: 210-211° C. IR: 3441, 3374, 3067, 3023, 2929, 2904, 2846, 2813, 1618, 1587, 1494, 1466, 1439, 1410, 1375, 1331, 1299, 1250, 1129, 1068, 1005, 983, 783, 765, 700. HRMS (ESI⁺): calc. for C₂₁H₂₇N₂O [M+H]⁺: 323.21179 found: 323.21146. ¹H NMR (400 MHZ, CDCL₃) δ7.33 (t, J=7.5 Hz, 2H, 2H-Ar), 7.28-7.19 (m, 3H, 3H-Ar), 6.98 (t, J=7.7 Hz, 1H, H-Ar), 6.56 (t, J=7.2 Hz, 2H, H-Ar), 4.34 (brs, 1H, OH), 3.93 (td, J=9.8, 6.1 Hz, 1H, CH), 3.62 (brs, 2H, NH₂), 3.13 (dd, J=15.6, 6.3 Hz, 1H, 1H of CH₂), 3.00 (d, J=11.2 Hz, 1H, CH₂), 2.93-2.73 (m, 5H, CH and 2 CH₂), 2.57 (tt, J=12.0, 4.1 Hz, 1H, CH), 2.47-2.32 (m, 2H, 2 H de 2 CH₂), 1.98-1.82 (m, 3H, CH₂ and H of CH₂), 1.75 (qd, J=12.3, 3.8 Hz, 1H, 1H de CH₂). ¹³C NMR (101 MHZ, CDCL₃) δ146.2 (Cq), 144.6 (Cq), 136.1 (Cq), 128.6(2CH), 127.0 (CH), 127.0 (2CH), 126.4 (CH), 119.5 (CH), 118.9 (Cq), 112.7 (CH), 66.2 (CH), 66.1 (CH), 53.8 (CH₂), 45.2 (CH₂), 43.1 (CH), 34.4 (CH₂), 34.0 (CH₂), 33.3 (CH₂), 26.6 (CH₂).

The regioisomer of interest 5-R1 trans was next entered into a Balz-Schiemann reaction to give the product (rac)-5-FBVM Trans with a yield of 59%. To separate the two enantiomers of (rac)-(+/−)-5-FBVM Trans, the latter was converted to a Mosher ester via commercial (R)-(+)-α-methoxy-α-trifluoromethylphenylacetic acid used as chiral copula. Under Steglich conditions, the two diastereoisomers 6-D₁ and 6-D₂ were obtained and separated with an overall yield of 60%.

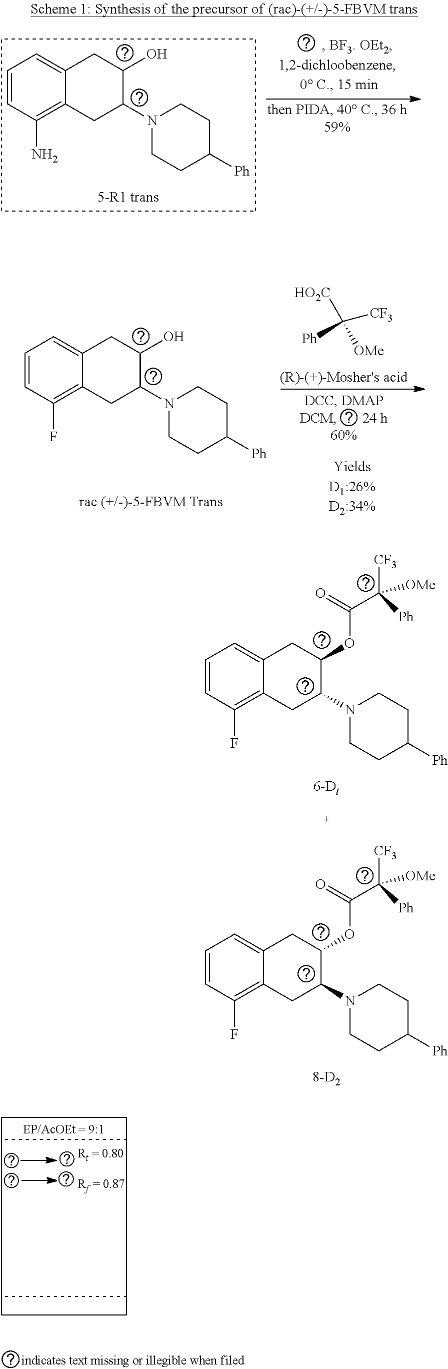 [SMILES: Nc1cccc2c1CC(N1CCC(c3ccccc3)CC1)C(O)C2 ; OC1Cc2cccc(F)c2CC1N1CCC(c2ccccc2)CC1 ; CO[C@@](C(=O)O)(c1ccccc1)C(F)(F)F ; CO[C@@](C(=O)O[C@@H]1Cc2cccc(F)c2C[C@H]1N1CCC(c2ccccc2)CC1)(c1ccccc1)C(F)(F)F ; CO[C@@](C(=O)O[C@H]1Cc2cccc(F)c2C[C@@H]1N1CCC(c2ccccc2)CC1)(c1ccccc1)C(F)(F)F]

5-Fluoro-3-(4-phenylpiperidin-1-yl)-1,2,3,4-tetrahydronaphthalen-2-ol ((rac)-(+/−)-5-FBVM):

In an oven-dried tubular reactor fitted with a stirrer and Teflon screw cap, compound 5-R1 (849 mg, 2.6 mmol), BF_{3·}Et₂O (818 μl, 6.6 mmol, 2.5 equivalents) and 1,2-dichlorobenzene (9 mL) were added. The mixture was cooled to 0° C., after which the addition was made of tert-BuONO (629 μl, 4.7 mmol, 1.8 equiv.) using a syringe under an argon atmosphere and at 0° C. The mixture was left under agitation at 0° C. for 15 min followed by the addition of a PIDA solution (170 mg, 0.5 mmol, 0.2 equiv.) in 1,2-dichlorobenzene (2 mL). The reactor was sealed and heated to 40° C. for 36h. After cooling, the mixture was evaporated in vacuo. The crude product was solubilised in DCM (30 mL) and washed with a saturated aqueous solution of NaHCO₃ (30 mL), dried over MgSO₄, then filtered under reduced pressure. The crude product obtained was purified by flash chromatography using EP/AcOEt (10:0 to 1:1) as eluent to obtain the desired (rac)-5-FBVM (500 mg, 59%) in the form of a pale beige solid. Mp: 147-148° C.

IR (v cm-1): 3434, 3079, 3048, 3025, 2931, 2910, 2870, 2840, 2813, 1579, 1492, 1462, 1411, 1376, 1278, 1238, 1141, 1124, 1072, 1003, 981, 782, 769, 711, 696. HRMS (ESI⁺): calc. for C₂₁H₂₅FNO [M+H]⁺: 326. 19146 found: 326.19131. ¹H NMR (250 MHZ, CDCL₃) δ7.37-7.17 (m, 5H, 5H-Ar), 7.11 (td, J=7.9, 5.7 Hz, 1H, H-Ar), 6.95-6.80 (m, 2H, 2 H-Ar), 4.34 (brs, 1H, OH), 3.87 (td, J=10.2, 5.6 Hz, 1H, CH), 3.33 (dd, J=16.2, 5.7 Hz, 1H, H de CH₂), 3.15-3.02 (m, 1H, H of CH₂), 3.02 -2.84 (m, 3H, CH of CH2 and CH2), 2.83-2.50 (m, 4H, 2H of CH2 and 2 CH), 2.44 (td, J=11.5, 2.5 Hz, 1H, H of CH₂), 2.01-1.86 (m, 3H, CH and H of CH₂), 1.85 -1.62 (m, 1H, H of CH₂). ¹³C NMR (63 MHZ, CDCL₃) δ161.1 (d, J=243.9 Hz, Cq), 146.2(Cq), 136.8 (d, J=4.3 Hz, Cq), 128.6 (2CH), 127.4 (d, J=8.7 Hz, CH), 127.0(2CH), 126.4 (CH), 124.7 (d, J=3.3 Hz, CH), 122.7 (d, J=17.6 Hz, Cq), 112.4 (d, J=21.9 Hz, CH), 66.2 (CH), 65.4(CH), 53.8(CH₂), 45.1(CH₂), 43.0 (CH), 37.9 (d, J=2.2 Hz, CH₂), 34.5 (CH₂), 34.0 (CH₂), 19.2 (d, J=3.3 Hz, CH₂). ¹⁹F NMR (235 MHz, CDCL₃) δ-117.3 (dd, J=9.9, 5.5 Hz).

### Synthesis of Mosher Esters 6-D1 and 6-D2:

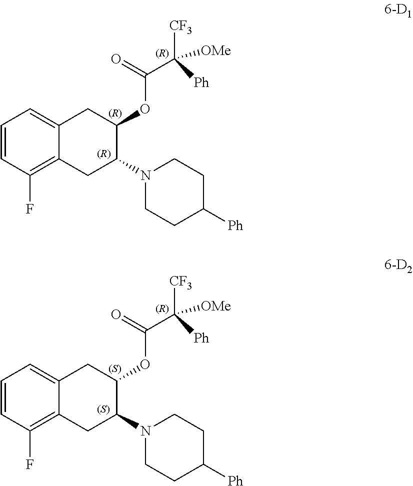

In an oven-dried round-bottom flask, rac-(+/−)-5-FBVM (650 mg, 2.0 mmol) was solubilised in DCM (5 mL). The addition was made of DCC (515 mg, 2.5 mmol, 1.25 equiv.), DMAP (5 mg, 0.4 mmol, 0.2 equiv.) and Mosher's acid (561 mg, 2.4 mmol, 1.2 equiv.) respectively under an argon atmosphere. The reaction mixture was left under agitation at ambient temperature for 24 hours. The reaction was halted by the addition of a saturated aqueous solution of NaHCO₃ (30 mL), then transferred to a separation funnel. The organic layer was collected and the aqueous layer was extracted with DCM (2×30 mL). The organic layers were then combined together, dried over MgSO₄ and filtered. The solvent was removed under reduced pressure. The crude product obtained was purified by flash chromatography on a silica gel column using the mixture EP/AcOEt (100:0 to 95:5) as eluent to obtain the desired products 6-D1 (276 mg, 26%) and 6-D2 (369 mg, 34%) in the form amorphous white solids.

(R)-(2R,3R)-5-Fluoro-3-(4-phenylpiperidin-1-yl)-1,2,3,4-tetrahydronaphthalen-2-yl 3,3,3-trifluoro-2-methoxy-2-phenylpropanoate (6-D1)

HRMS (ESI⁺): calc. for C₃₁H₃₂F₄NO₃ [M+H]⁺: 542.23128 found: 542.23097. IR (v cm⁻¹): 3060.5, 3002.6, 2975.1, 2947.0, 2889.8, 2850.1, 2353.1, 1738.2, 1588.1, 1496.7, 1474.7, 1452.4, 1405.2, 1384.4, 1258.2, 1197.8, 1187.1, 1170.0, 1112.5, 1079.8, 1054.2, 1015.7, 995.1, 969.9, 952.5, 88.8, 793.5, 763.0, 745.8, 736.7, 720.8, 704.0, 661.8. ¹H NMR (250 MHz, CDCL₃) δ7.80 (dd, J=6.5, 3.1 Hz, 2H, 2H-Ar), 7.54 -7.44 (m, 3H, 3 H-Ar), 7.41-7.31 (m, 2H, 2H-Ar), 7.31-7.21 (m, 3H, 3 H-Ar), 7.21-7.09 (m, 1H, 1 H-Ar), 7.02-6.80 (m, 2H, 2 H-Ar), 5.63 (td, J=9.5, 5.9 Hz, 1H, CH), 3.78 (s, 3H, OMe), 3.32-3.09 (m, 4H, CH and 3 H of 3CH₂), 3.07-2.76 (m, 4H,2H of 2CH₂ and CH₂), 2.64-2.40 (m, 2H, CH and 1H of CH₂), 2.02-1.62 (m, 4H, 2 CH₂). ¹³C NMR (63 MHZ, CDCL₃) δ166.0 (Cq), 160.7 (d, J=244.5 Hz, Cq), 146.5 (Cq), 135.7 (d, J=4.4 Hz, Cq), 132.6 (Cq), 129.8 (CH), 128.6 (4CH), 127.8 (2CH), 127.5 (d, J=8.6 Hz, CH), 126.9 (2CH), 126.3 (CH), 124.0 (d, J=3.3 Hz, CH), 123.5 (q, J=288.9, CF₃), 122.5 (d, J=17.7 Hz, Cq), 112.9 (d, J=21.8 Hz, CH), 85.0 (q, J=27.4 Hz, Cq), 71.2 (CH), 63.2 (CH), 55.70 (d, J=1.5 Hz, CH₃), 52.8 (CH₂), 46.8 (CH₂), 42.9 (CH), 34.8 (d, J=2.1 Hz, CH₂), 34.5 (CH₂), 33.2 (CH₂), 19.9 (d, J=2.9 Hz, CH₂). ¹⁹F NMR (235 MHZ, CDCL₃) δ-71.1, -117.8 (dd, J=9.4, 5.7 Hz).

(R)-(2S,3S)-5-Fluoro-3-(4-phenylpiperidin-1-yl)-1,2,3,4-tetrahydronaphthalen-2-yl 3,3,3-trifluoro-2-methoxy-2- phenylpropanoate (6-D2):

HRMS (ESI⁺): calc. for C₃₁H₃₂F₄NO₃ [M+H]⁺: 542.23128 found: 542.23125. IR (v cm⁻¹): 3025.6, 2978.2, 2943.6, 2911.9, 2849.8, 2794.8, 2737.6, 2621.6, 2603.5, 2497.9, 1746.9, 1619.3, 1584.5, 1493.1, 1465.6, 1450.7, 1397.5, 1385.3, 1325.9, 1293.4, 1243.5, 1187.3, 1166.1, 1122.5, 1108.9, 1079.6, 1012.2, 966.5, 786.5, 762.8, 734.5, 719.2, 699.7. ¹H NMR (250 MHz, CDCL₃) δ7.74-7.61 (m, 2H, 2H-Ar), 7.47 -7.38 (m, 3H, 3H-Ar), 7.34-7.26 (m, 2H, 2H-Ar), 7.23-7.06 (m, 4H, 4H-Ar), 6.94 -6.83 (m, 2H, 2H-Ar), 5.51 (td, J=8.6, 5.5 Hz, 1H, CH), 3.63 (q, J=1.2 Hz, 3H, OMe), 3.34 (dd, J=16.2, 5.6 Hz, 1H, H of CH₂), 3.15-2.73 (m, 6H, CH₂, 3H of 3CH₂ and CH), 2.51 (td, J=11.3, 2.7 Hz, 1H, H of CH₂), 2.42-2.22 (m, 2H, H of CH₂ and CH), 1.67 (dtq, J=28.9, 12.2, 4.0 Hz, 4H, 2CH₂). ¹³C NMR (63 MHZ, CDCL₃) δ166.0 (Cq), 160.7 (d, J=244.8 Hz, Cq), 146.5 (Cq), 135.6 (d, J=4.4 Hz, Cq), 132.4 (Cq), 129.7 (CH), 128.5 (2CH), 128.5 (2CH), 127.8 (2CH), 127.5 (d, J=8.6 Hz, CH), 126.9 (2CH), 126.2 (CH), 124.0 (d, J=3.4 Hz, CH), 123.5 (q, J=288.6 Hz, CF₃), 122.7 (d, J=17.7 Hz, Cq), 113.0 (d, J=21.7 Hz, CH), 84.7 (q, J=27.5 Hz, Cq), 72.3 (CH), 62.6 (CH₂), 55.7 (CH₃), 51.3 (CH₂), 48.7 (CH₂), 42.8 (CH), 34.7 (d, J=2.2 Hz, CH₂), 34.2 (CH), 33.6 (CH₂), 21.3 (d, J=3.1 Hz, CH₂). ¹⁹F NMR (235 MHZ, CDCL₃) δ-71.84, -118.06 (dd, J=9.5, 5.7 Hz).

1-((2R,3R)-8-Fluoro-3-(((R)-3,3,3-trifluoro-2-methoxy-2-phenylpropanoyl)oxy)-1,2,3,4-tetrahydronaphthalen-2-yl)-4- phenylpiperidin-1-ium chloride (6-D1.HCl)

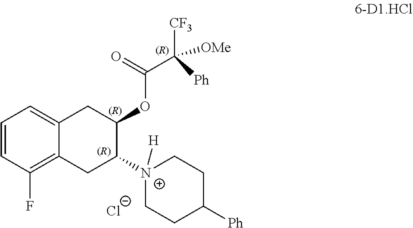

In a round-bottom flask, compound 6-D1 (250 mg, 0.46 mmol) was solubilised in a dioxane (3 ml) and cooled to 0° C. A 4 N solution of HCl in dioxane (173 μL, 0.69 mmol, 1.5 equivalent) was added dropwise and the mixture left under agitation at ambient temperature for 24 hours. The solvent and excess HCl were removed under atmospheric pressure to obtain crystals. The crude product was triturated with Et₂O to obtain a white solid which was collected by filtration and dried in vacuo to obtain 6-D1-HCl with a quantitative yield. Some crystals were collected for X-ray analysis. Mp: 168-169° C. IR (v cm⁻¹): 3028.3, 3006.8, 2951.6, 2853.6, 2359.9, 1748.3, 1590.4, 1470.3, 1446.9, 1245.0, 1163.4, 1118.8, 1078.9, 1014.8, 963.9, 845.0, 750.2, 721.3, 700.1, 664.1, 552.4. ¹H NMR (250 MHZ, DMSO-d₆) δ11.62 (bs, 1H, NH), 7.48 (s, 5H, 5H-Ar), 7.40-7.30 (m, 2H, 2H-Ar), 7.29-7.12 (m, 4H, 4 H-Ar), 7.10-6.92 (m, 2H, 2H-Ar), 5.95 (d, J=4.9 Hz, 1H, CH), 3.99-3.83 (m, 1H, CH), 3.79-2.90 (m, 11H, 4CH₂ and OMe), 2.88-2.67 (m, 1H, CH), 2.27 (d, J=14.0 Hz, 2H, 2H of 2 CH₂), 2.07-1.86 (m, 2H, 2H of 2 CH₂). ¹³C NMR (63 MHZ, DMSO-d₆) δ164.7 (Cq), 159.1 (d, J=244.2 Hz, Cq), 144.1 (Cq), 135.5 (d, J=2.9 Hz, Cq), 130.7 (Cq), 130.0 (CH), 128.8 (2CH), 128.6 (2CH), 128.2 (d, J=8.2 Hz, CH), 127.2 (2CH), 126.6 (3CH), 124.2 (CH), 123.1 (q, J=290.4 Hz, CF₃) 119.7 (d, J=16.5 Hz, Cq), 113.4 (d, J=20.9 Hz, CH), 84.2 (q, J=27.2, 26.6 Hz, Cq), 71.9 (CH), 63.14 (CH), 55.6 (CH₃), 50,6 (CH₂), 49.3 (CH₂), 39.2 (CH), 32.6 CH₂, 29.8 (CH₂), 29.6 (CH2), 20.7 (CH₂). ¹⁹F NMR (235 MHz, DMSO-d₆) δ-70.9, -119.9 (dd, J=9.1, 5.8 Hz).

Crystallisation of the Mosher esters 6-D₁ and 6-D₂ in various solvents and combination of solvents failed. However, the solubilisation of 6-D₁ in a 4 N HCl solution in dioxane, followed by slow evaporation of the solvent at ambient temperature allowed the obtaining of crystals of the salt 6-D₁·HCl. Analysis of the latter by X-ray diffraction showed that the three asymmetric centres of 6-D₁ have the absolute stereochemistry (R, R, R).

The crystals were entered into a hydrolysis reaction of the ester in a basic medium to give (R,R)-5-FBVM with a yield of 80%. Finally, (R,R)-5-FBVM was analysed by polarimetry to determine the direction of deviation of polarised light. This analysis showed a rotatory power of [α_{D}]^{20° C.}=−82.0° (10 mg in 1 ml of Chloroform). The product (−)-(R,R)-5-FBVM was therefore obtained (Scheme 3).

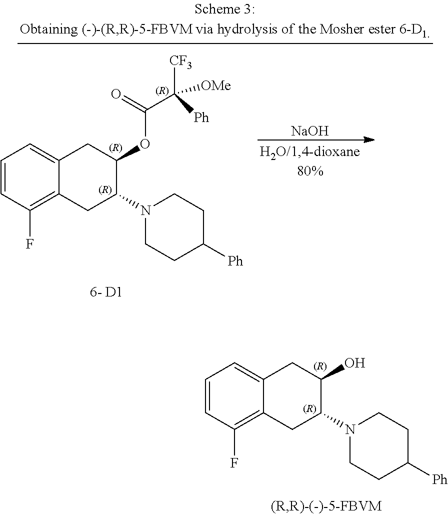

(2R,3R)-5-Fluoro-3-(4-phenylpiperidin-1-yl)-1,2,3,4-tetrahydronaphthalen-2-ol (R,R)-(−)-5-FBVM)

In a round-bottom flask, compound 6-D1 (96 mg, 0.14 mmol) was solubilised in 1,4-dioxane (3 mL), after which an aqueous 1 M solution of NaOH (3 mL) was added and the reaction mixture was left under agitation at 50° C. for 18 hours. The solvents were removed under reduced pressure and the crude product obtained was solubilised in water and extracted with AcOEt (3×5 mL). The organic phases were combined and dried over MgSO₄. The solvent was removed under reduced pressure to obtain the desired product (R,R)-(−)-5-FBVM in the form of a pale beige solid (48 mg, 83%). [α_{D}]^{20° C.}=−82.0 (10 mg in 1 mL of Chloroform). Mp: 148-149° C. IR (v cm⁻¹): 3379, 3062, 3027, 2937, 2919, 2848, 2798, 2771, 1618, 1601, 1578, 1493, 1464, 1453, 1443, 1413, 1392, 1374, 1337, 1325, 1309, 1285, 1238, 1218, 1161, 1137, 1074, 1052, 1022, 1003, 977, 804, 791, 771, 758, 710, 701. HRMS (ESI⁺): calc. for C₂₁H₂₅FNO [M+H]⁺: 326. 19146 found: 326.19131. ¹H NMR (250 MHZ, CDCL3) δ7.37-7.17 (m, 5H, 5H-Ar), 7.11 (td, J=7.9, 5.7 Hz, 1H, H-Ar), 6.95-6.80 (m, 2H, 2 H-Ar), 4.34 (brs, 1H, OH), 3.87 (td, J=10.2, 5.6 Hz, 1H, CH), 3.33 (dd, J=16.2, 5.7 Hz, 1H, H of CH₂), 3.15-3.02 (m, 1H, H of CH₂), 3.02-2.84 (m, 3H, CH of CH2 and CH2), 2.83-2.50 (m, 4H, 2H of CH2 and 2 CH), 2.44 (td, J=11.5, 2.5 Hz, 1H, H of CH₂), 2.01-1.86 (m, 3H, CH and H of CH₂), 1.85-1.62 (m, 1H, H of CH₂). ¹³C NMR (63 MHZ, CDCL₃) δ161.1 (d, J=243.9 Hz, Cq), 146.2(Cq), 136.8 (d, J=4.3 Hz, Cq), 128.6 (2CH), 127.4 (d, J=8.7 Hz, CH), 127.0(2CH), 126.4 (CH), 124.7 (d, J=3.3 Hz, CH), 122.7 (d, J=17.6 Hz, Cq), 112.4 (d, J=21.9 Hz, CH), 66.2 (CH), 65.4(CH), 53.8(CH₂), 45.1(CH₂), 43.0 (CH), 37.9 (d, J=2.2 Hz, CH₂), 34.5 (CH₂), 34.0 (CH₂), 19.2 (d, J=3.3 Hz, CH₂). ¹⁹F NMR (235 MHZ, CDCL₃) δ-117.3 (dd, J=9.9, 5.5 Hz).

Analysis of the latter by chiral HPLC showed that it is effectively a single enantiomer.

Similarly, the Mosher ester 6-D₂ was hydrolysed to give (+)-(S,S)-5-FBVM with a yield of 89%. This was analysed by polarimetry and exhibited a rotatory power of [α_{D}]^{20° C.}=+82.8° (10 mg in 1 mL of Chloroform). Chiral HPLC analysis thereof under the same conditions showed that it is a single enantiomer with a longer retention time.

(2S,3S)-5-Fluoro-3-(4-phenylpiperidin-1-yl)-1,2,3,4-tetrahydronaphthalen-2-ol ((S,S)-(+)-5-FBVM)

In a round-bottom flask, compound 6-D2 (100 mg, 0.17 mmol) was solubilised in 1,4-dioxane (3 mL), after which a 1 M aqueous solution of NaOH (3 mL) was added and the reaction mixture left under agitation at 50° C. for 18 hours. The solvents were removed under reduced pressure and the crude product obtained was solubilised in water and extracted with AcOEt (3×5 ml). The organic phases were combined and dried over MgSO4. The solvent was removed under reduced pressure to obtain the desired product (S,S)-(+)-5-FBVM in the form of a pale beige solid (56 mg, 99%). [α_{D}]^{20° C.}=+82.8 (10 mg in 1 mL of chloroform).

Mp:148-149° C. IR (v cm⁻¹): 3379, 3064, 3027, 2937, 2926, 2849, 2798, 2771, 1619, 1601, 1578, 1492, 1463, 1453, 1443,1413, 1392, 1374, 1337, 1325, 1309, 1285, 1238, 1218, 1161, 1137, 1073, 1052, 1022, 1003, 791, 772, 758, 711, 701. HRMS (ESI+): calc. for C₂₁H₂₅FNO [M+H]⁺: 326. 19146 found: 326.19131. ¹H NMR (250 MHZ, CDCL₃) δ7.37-7.17 (m, 5H, 5H-Ar), 7.11 (td, J=7.9, 5.7 Hz, 1H, H-Ar), 6.95-6.80 (m, 2H, 2 H-Ar), 4.34 (brs, 1H, OH), 3.87 (td, J=10.2, 5.6 Hz, 1H, CH), 3.33 (dd, J=16.2, 5.7 Hz, 1H, H of CH₂), 3.15-3.02 (m, 1H, H of CH₂), 3.02-2.84 (m, 3H, CH of CH2 and CH2), 2.83-2.50 (m, 4H, 2H of CH2 and 2 CH), 2.44 (td, J=11.5, 2.5 Hz, 1H, H of CH₂), 2.01-1.86 (m, 3H, CH and H of CH₂), 1.85-1.62 (m, 1H, H of CH₂). ¹³C NMR (63 MHZ, CDCL₃) δ161.1 (d, J=243.9 Hz, Cq), 146.2(Cq), 136.8 (d, J=4.3 Hz, Cq), 128.6 (2CH), 127.4 (d, J=8.7 Hz, CH), 127.0(2CH), 126.4 (CH), 124.7 (d, J=3.3 Hz, CH), 122.7 (d, J=17.6 Hz, Cq), 112.4 (d, J=21.9 Hz, CH), 66.2 (CH), 65.4(CH), 53.8(CH₂), 45.1(CH₂), 43.0 (CH), 37.9 (d, J=2.2 Hz, CH₂), 34.5 (CH₂), 34.0 (CH₂), 19.2 (d, J=3.3 Hz, CH₂). ¹⁹F NMR (235 MHZ, CDCL₃) δ-117.3 (dd, J=9.9, 5.5 Hz).

### Synthesis of the Boronic Ester 8 (Precursor for ¹⁸F Radiolabelling)

To obtain ¹⁸F radiolabelling, the boronic ester 8 was used as precursor (corresponding to the above-mentioned compound of formula (III)). This was synthesised following the synthesis route described in Scheme 4. The compound 5-R₁ Trans was converted in-situ to a corresponding diazonium salt via action of NaNO₂ in the presence of PTSA monohydrate. This led to the iodine intermediate 7 Trans under the action of potassium iodide contained in the reaction medium, with a yield of 50%. The conditions of Miyaura borylation subsequently allowed conversion of the intermediate 7 Trans to the desired compound 8 Trans with a yield of 44%.

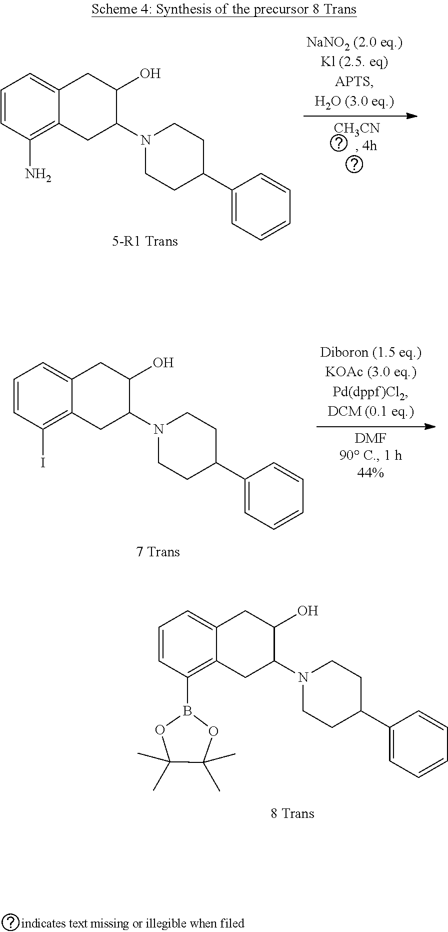 [SMILES: Nc1cccc2c1CC(N1CCC(c3ccccc3)CC1)C(O)C2 ; OC1Cc2cccc(I)c2CC1N1CCC(c2ccccc2)CC1 ; CC1(C)OB(c2cccc3c2CC(N2CCC(c4ccccc4)CC2)C(O)C3)OC1(C)C]

5-lodo-3-(4-phenylpiperidin-1-yl)-1,2,3,4-tetrahydronaphthalen-2-ol (7)

In a round-bottom flask, compound 5-R1 (1.00 g, 3.10 mmol) was solubilised in CH₃CN (18 ml), after which PTSA·H₂O (1.80 g, 9.32 mmol, 3.0 equivalents) was added in a single addition. The mixture was cooled to 0° C., and the addition was made of a solution of NaNO₂ (428 mg, 6.2 mmol, 2.0 equivalents) and KI (1.20 g, 7.75 mmol, 2.5 equivalents) in water (5 mL). After being left under agitation at 0° C. for 15 minutes, the reaction mixture was heated to room temperature and left under agitation for 4 h. Water (17 mL) and a saturated NaHCO₃ solution (35 mL) were successively added. The aqueous layer was extracted with AcOEt (3×25 ml) and the combined organic phases were dried over MgSO_{4,} filtered and dried under reduced pressure. The residue was purified by flash chromatography on a silica gel column (PE/AcOEt: 85:15) to obtain the desired product 7 in the form of a yellowish solid (587 mg, 50%).

R_{f}: 0.43. 1H NMR (250 MHZ, CDCL₃) ppm: 7.70 (d, J=7.8 Hz, 1H, 1 H-Ar), 7.39-7.16 (m, 6H, 6 H-Ar), 7.10 (d, J=7.7 Hz, 1H, 1 H-Ar), 6.83 (t, J=7.7 Hz, 1H, OH), 3.83 (td, J=10.2, 5.6 Hz, 1H, CH), 3.24 -2.33 (m, 12H, 6 CH₂).

3-(4-Phenylpiperidin-1-yl)-5-(4,4,5,5-tetramethyl-1,3,2-dioxaborolan-2-yl)-1,2,3,4-tetrahydronaphthalen-2-ol (8):

In an oven-dried tube, compound 7 (100 mg, 0.23 mmol) was solubilised in degassed anhydrous DMF (2 mL), and B2Pin₂ (88 mg, 0.35 mmol, 1.5 equivalent), KOAc (68 mg, 0.69 mmol, 3.0 equivalents) and Pd(dppf)Cl₂·DCM (19 mg, 0.02 mmol, 0.1 equivalent) were respectively added thereto. The tube was sealed under argon and heated to 160° C for 1 h 30. The reaction mixture was cooled to ambient temperature after which the DMF was dried under reduced pressure. The residue was washed with brine (20 ml) and filtered on a celite buffer. The filtrate was extracted with AcOEt (3×10 mL). The organic phases were combined and dried over MgSO₄. After removing the solvent under reduced pressure, the residue was purified by flash chromatography on a silica gel column (PE/AcOEt: 85:15 and 5% Et₃N) to obtain the desired product 8 in the form of a white solid (50 mg, 44%). R_{f}: 0.27. ¹H NMR (250 MHz, DMSO) ppm: 7.48-7.43 (m, 1H, H-Ar), 7.33-7.23 (m, 4H, 4 H-Ar), 7.22-7.15 (m, 3H, 3 H-Ar), 4.49 (s, 1H, OH), 3.83 (d, J=7.8 Hz, 1H, CH), 2.99 (ddd, J=32.1, 20.4, 8.7 Hz, 4H, 2 CH₂), 1.81-1.63 (m, 4H, 2 CH₂), 1.32 (s, 12H, 4 CH₃).

### Radiochemistry

Beforehand, radiochemistry had allowed the preparation of rac (+/−)-5-FBVM Trans labelled with Fluorine 18, and chiral separation under heat of the two isomers contained therein. These are the 2 enantiomers labelled with ¹⁸F and called ¹⁸F E1 and ¹⁸F E2 (Scheme 5).

Since all chiral HPLC analyses via UV detection (cold chemistry) and radio detection (hot chemistry) were performed on the same Chiralpak IA support, we can therefore assert through combination of these results that the hot isomer ¹⁸F E1 corresponds to the cold isomer (−)-(R,R)-5 FBVM, and ¹⁸F E2 to (+)-(S,S)-5-FBVM.

According to chiral radio-analytical HPLC analysis, the retention time for (−)-(R,R)-5-[¹⁸F]FBVM=¹⁸F E1 is 16 minutes, and the retention time for (+)-(S,S)-5-[¹⁸F]FBVM=¹⁸F E2 is 21 minutes.

Via rebound effect, it can be concluded that there are 3 molecules labelled with ¹⁸F:
the most active and able to be used as ¹⁸F tracer, which is the hot derivative (−)(R,R)-5-[F]FBVM=¹⁸F E1 (corresponding to the above-mentioned compound of formula (I)),
the other diastereoisomer (+)-(S,S)-5-[¹⁸F]FBVM=¹⁸F E2 (corresponding to the above-mentioned compound of formula (II)); and
the mixture of both: (+/−)-5-[¹⁸F]FBVM Trans.

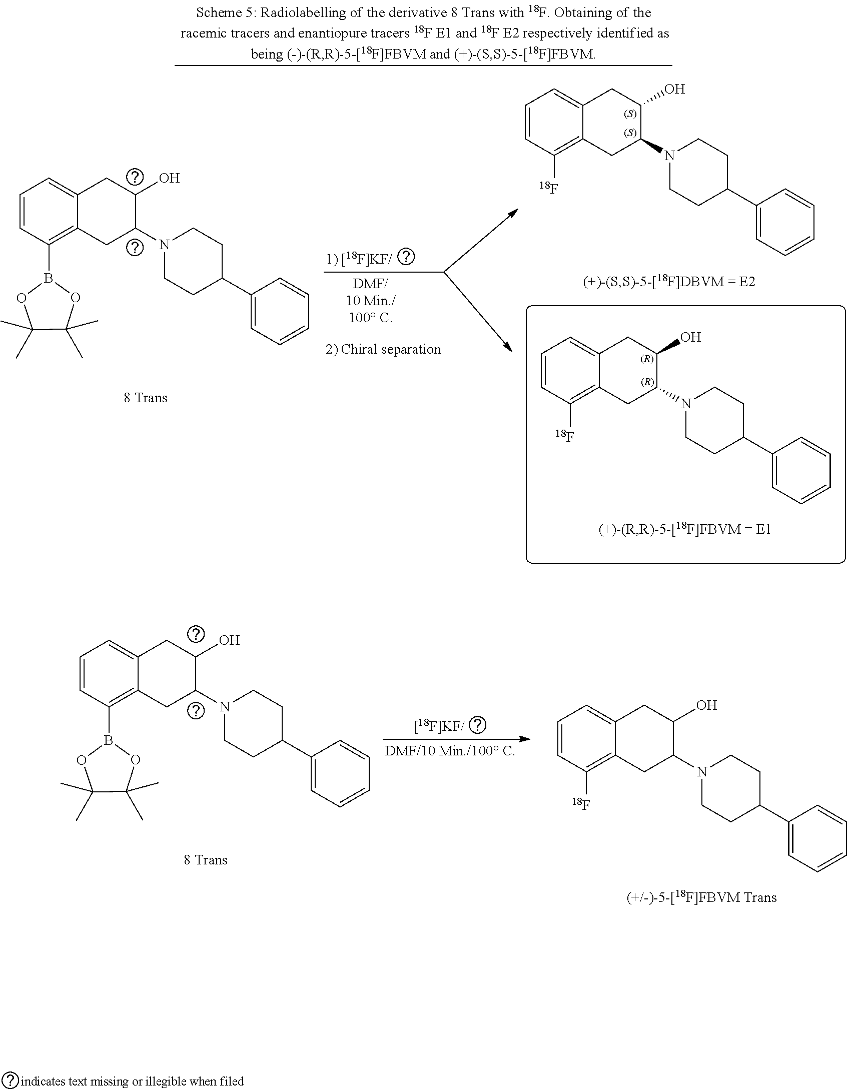

### Radiosynthesis of rac-¹⁸F-5-FBVM, (R,R)-(−)- ¹⁸F-5-FBVM and (S,S)-(+)-¹⁸F-5-FBVM

### Production of the Radiotracers

### Production of (+/−) [¹⁸F]-5-FBVM

The fluoride ions [¹⁸F] were produced using a cyclotron (PET trace, GE Healthcare) by irradiation of a water target enriched with oxygen18 by a proton beam by means of the nuclear reaction ₁₈O(p,n)¹⁸F. The produced fluoride ions [¹⁸F] were transferred to a modified automated system: TRACERlab FX-FN Pro (GE), and loaded onto an anion exchange cartridge (Waters Sep-Pak Accell Light QMA conditioned with potassium carbonate solution). The trapped fluoride ions [¹⁸F] were eluted from the cartridge with 550 μL of a solution containing KOTf (5 mg) and K₂CO₃ (50 μg). Azeotropic distillation was afterwards carried out with the addition of 1 mL acetonitrile. The water was evaporated at 90° C. under a flow of helium and low pressure, and this operation was repeated twice before performing nucleophilic substitution. The precursor 8 (2 mg) together with Cu(OTf)₂ (3.6 mg) dissolved in DMF (960 μL) and pyridine (40 μL) were added to the fluoride ions [¹⁸F]. The mixture was brought to 100° C. for 10 min, then cooled to 30° C. and diluted with water (8 mL). The reaction mixture was loaded onto a tC18 plus cartridge (Waters) and rinsed with water (4 mL) to trap the product of interest and remove most of the polar compounds. The product (+/−) [¹⁸F]-5-FBVM was eluted from the cartridge with acetonitrile (2 mL) and the solution diluted with 0.1 M ammonium acetate (1 mL). The solution obtained was loaded onto the HPLC injection loop of the automated system and purified on a semi-preparative column (PhenylHexyl-Phenomenex, 10×250 mm) using as mobile phase a mixture of ACN/0.1M ammonium acetate 70:30 at a flow rate of 4 mL·min⁻¹. Under these conditions, (+/−) [¹⁸F]-5-FBVM was collected with a retention time of about 11 min. The collected fraction was diluted with water (30 mL) and loaded onto a tC18 light cartridge (Waters), and the cartridge was rinsed with water (5 mL). The (+/−) [¹⁸F]-5-FBVM was eluted from the cartridge using injectable ethanol (0.8 mL) and the formulation was completed with the addition of 7.2 mL NaCl (0.9%).

Production of (−) [¹⁸F]-5-FBVM and (+) [¹⁸F]-5-FBVM

Production of the isolated enantiomers (+) [¹⁸F]-5-FBVM and (−) [¹⁸F]-5-FBVM took place following the production method of (+/−) [¹⁸F]-5-FBVM with the addition of HPLC chiral purification.

The collected pure fraction of (+/−) [¹⁸F]-5-FBVM was loaded onto the HPLC loop of the automated system to carry out further HPLC purification, this time on a chiral semi-preparative column (Chiralpak IA, Daicel, 10×250 mm). The mobile phase used was composed of acetonitrile/0.1 M ammonium acetate: 85:15, and purification was performed at 4 mL·min⁻¹. Under these conditions, (−) [¹⁸F]-5-FBVM was collected with a retention time of about 18 min, while (+) [¹⁸F]-5-FBVM exhibited a retention time of about 22 min. With this method, each pure enantiomer can be produced separately. The collected enantiomerically pure fraction (−) or (+) [¹⁸F]-5-FBV, was diluted in water (30 mL) and loaded onto a tC18 light cartridge (Waters). The cartridge was rinsed with water (5 mL). (−) [¹⁸F]-5-FBVM or (+) [¹⁸F]-5-FBVM were eluted from the cartridge with injectable ethanol (0.8 mL) and the formulation was completed with the addition of 7.2 mL NaCl (0.9%).

Production of (−)-(R,R)-[¹⁸F]-5-FBVM with a Single HPLC Purification

Since the most active identified compound is (−)-(R,R)-[¹⁸F]-5-FBVM and it corresponds to the first compound eluted at HPLC chiral purification, the production method was modified so as only to have one single HPLC purification.

The method reproduces the one described above up as far as the purification step. The crude solution of (+/−) [¹⁸F]-5-FBVM was loaded onto the HPLC loop and injected into the chiral column (Chiralpak IA, Daicel, 10×250 mm). Purification was conducted using a mixture of acetonitrile/methanol/0.1 M ammonium acetate: 70:10:20 as mobile phase, and at a flow rate 4 mL/min. Under these conditions, the retention time of (−)-(R,R)-[¹⁸F]-5-FBVM was about 14.5 min. The formulation was completed following the above-described methods.

### Quality Control of the Radiotracers

The radiotracers were controlled by analytical HPLC equipped with a UV and radio detector. Purity of (+/−) [¹⁸F]-5-FBVM was verified on an analytical column (Phenomenex Luna 5 μ Phenyl Hexyl 4.6×250 mm) using ACN/0.1 M ammonium acetate 70:30 as mobile phase and a flow rate of 1 mL/min. Under these conditions, the retention time was 9 min.

For enantiomeric purification, in addition to the preceding control and HPLC control using a chiral column (Chiralpak IA 4.6×250 mm 5 μ), it is possible to evaluate the enantiomeric purity of (+) [¹⁸F]-5-FBVM or (−) [¹⁸F]-5-FBVM. For this control, ACN/0.1 M ammonium acetate 85:15 was used as mobile phase at a flow rate of 1 ml/min. Under these conditions, the retention time of (−) [¹⁸F]-5-FBVM or (+) [¹⁸F]-5-FBVM was 16.2 and 21.1 min respectively.

For all compounds, the radiochemical purity was higher than 99%, molar activity was higher than 100 GBq/μmole, and no degradation was observed in the formulation media or in the serum for at least 4 h.

### Biology

### In Vitro Biology

Measurement of the affinity of the different compounds for VAChT was obtained by radioligand binding method on rat brain membrane preparation, following the method described in Scheunemann et al. (Bioorg Med Chem 2004;12:1459-65). The L-(−)-vesamicol was supplied by Sigma Aldrich (Saint-Quentin-Fallavier, France) and [³H]vesamicol (specific activity 1705.7 GBq/mmol) by Perkin-Elmer (Courtaboeuf, France). The IC₅₀ values were determined graphically for each compound and Ki (inhibition constant) was calculated following the method of Cheng & Prussoff (Biochemical pharmacology 1973;22:3099-108). The results are expressed as a mean of Ki values±standard deviation from the mean after 3 independent experiments.

By complementarity, assigning of the values of the affinities of the three cold fluorinated products was:
rac (+/−)-5-FBVM Trans: Ki=1.3+/−0.2 nM
(−)-(R,R)-5-FBVM E1: Ki=0.9+/−0.3 nM
(+)-(S,S)-5-FBVM. E2: Ki=35+/−5 nM

In comparison, the Ki of (−)-FEOBV is: Ki=61±2.8 nM under the same experimental conditions.

### In Vivo Biology

### Conducted Experimental Demonstrations

The present invention, based on the structure of molecules of (benzo)vesamicol type, allows a good level of in vitro specificity to be reached, and the first radioligand (rac (+/−)-5-FBVM Trans) exhibited its potential as in vivo PET tracer in the rat having regard to excellent passing thereof through the blood-brain barrier, to specific accumulation thereof in the cerebral regions where the VAChT are located, and to the good in vivo stability thereof.

In addition, the 2 isomers of racemic [¹⁸F]FBVM were separated, thereby obtaining (−)-(R,R)-5-FBVM ¹⁸F E1 and (+)-(S,S)-5-[18 F]FBVM ¹⁸F E2. It was shown that (−)-(R,R)-5-FBVM ¹⁸F E1 performs better, in terms of passing the blood-brain barrier and binding to VAChT, than the racemic and the other isomer.

In the striatum, the cerebral region known to have a denser concentration of VAChT, the percentage of tracer dose injected per gram of tissue (% ID/g) is highest for (−)-(R,R)-5-FBVM ¹⁸F E1 (Table 1). The values obtained for the accumulation ratios between the striatum and the cerebellum, known to be a zone of reference for non-specific fixation since devoid of VAChT, also demonstrate the superiority of (+)-(S,S)-5-[¹⁸F]FBVM ¹⁸F E2 over the other isomer (−)-(R,R)-5-FBVM ¹⁸F E1, and over the racemic [¹⁸F]FBVM.

TABLE 1
Cerebral accumulation of the tracers in the rat

| | | | Striatum/ |
|---|---|---|---|
| | Cerebellum | Striatum | cerebellum |
| [¹⁸F]FBVM | 0.185 ± 0.003 | 0.721 ± 0.011 | 3.90 |
| (−)-(R,R)-5-FBVM | 0.232 ± 0.017 | 1.356 ± 0.069 | 5.84 |
| ¹⁸F E1 | | | |
| (+)-(S,S)-5-[¹⁸F]FBVM | 0.215 ± 0.011 | 0.327 ± 0.015 | 1.52 |
| ¹⁸F E2 | | | |

The results are expressed as a percentage of injected dose/g of cerebral tissue (% ID/g)±SEM, 2 hours after i. v. injection of the tracers; n=6/group.

It was also shown that cerebral fixation of (−)-(R,R)-5-FBVM ¹⁸F E1 is indeed specific to VAChT, since it is strongly inhibited in animals in which these sites were occupied by administering a known ligand of VAChT. In the striatum, the accumulation of (−)-(R,R)-5-FBVM ¹⁸F E1 was effectively reduced by 46% in animals having received a dose of (−)vesamicol of 0.5 μmole/kg, 5 minutes before injection of the tracer.

At a second step, with the same experimental protocol as in the rat, the inventors compared (−)-(R,R)-5-FBVM ¹⁸F E1 with the tracer described in the literature: [¹⁸F]FEOBV, for which radiolabelling was performed on site.

The results (Table 2) show a stronger accumulation of (−)-(R,R)-5-FBVM ¹⁸F E1 compared with [¹⁸F]FEOBV in the striatum, with a higher signal-to-noise ratio (=striatum/cerebellum) for (−)-(R,R)-5-FBVM E1 than for [¹⁸F]FEOBV.

TABLE 2
Cerebral accumulation of
(−)-(R,R)-5-FBVM E1 and of [¹⁸F]FEOBV

| | | | Striatum/ |
|---|---|---|---|
| | Cerebellum | Striatum | cerebellum |
| (−)-(R,R)-5-FBVM | 0.232 ± 0.017 | 1.356 ± 0.069 | 5.84 |
| ¹⁸F E1 | | | |
| [¹⁸F]FEOBV | 0.844 ± 0.025 | 0.235 ± 0.011 | 3.59 |

The results are expressed as a percentage of injected dose/g of cerebral tissue (% ID/g)±SEM, 2 hours after i.v. injection of the tracers; n=6/group.
In Vivo Operating Modes of rac-5-FBVM, (R,R)-(−)-5FBVM and (S,S)-(+)-5FBVM

Animals: The experiments were conducted in male rates of Wistar strain weighing 250-300 g (Breeding Centre R. Janvier, Le Genest St Isle, France). All procedures were carried out paying heed to European regulations on animal experimentation (2010/63/EU) with authorisation from the regional ethics committee on animal experiments: Comité Régional d'Ethique en Expérimentation Animale.

Biodistribution studies: The rats in the control group were given an i.v. injection of tracer (4-6 MBq in 0.3 mL) under isoflurane gas anaesthesia (n=6/group). In the VES group (n=6/group), injection of the tracer was preceded (5 min) by an i.v. injection of vesamicol (0.5 μmol/kg). The rats were sacrificed by decapitation 2 h after injection of the tracer. The entire brain was sampled and dissected in different regions: cortex, striatum, hippocampus, thalamus and cerebellum. Blood and bone fractions were also sampled. The biological samples were weighed, the radioactivity thereof was measured with a γ counter (2480 Gamma counter Wizard, Perkin Elmer), and the percentage of injected dose/g of tissue (% ID/g) was calculated.

PET imaging: The rats received an i.v. injection of the tracer of 37 MBq. Acquisitions were obtained with a microPET explore VISTA-CT imaging system (GE Healthcare, France) under isoflurane anaesthesia (Baxter, France), at 4-5% in oxygen for induction, followed by 1.5-2% during recording. Each acquisition lasted 91 minutes and the images were sequenced in list-mode with 1 sequence of 1 min followed by 9 sequences s of 10 min. After correction for attenuation, the images were reconstructed using a 2-D OSEM algorithm (GE Healthcare, France) in voxels of 0.3875×0.3875×0.775 mm³.

1. A compound having the following formula (I):

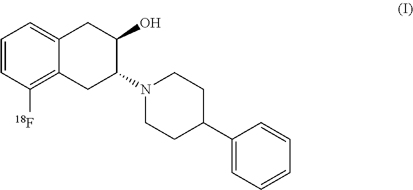 [SMILES: O[C@@H]1Cc2cccc([18F])c2C[C@H]1N1CCC(c2ccccc2)CC1]

2. (canceled)

3. An in vivo method of diagnosing a cholinergic neurodegenerative disease in a subject in need thereof, comprising administering the compound of claim 1 to the subject and detecting accumulation of the compound in vesicular acetylcholine transporter (VAChT) in the endings of the cholinergic neurons in the brain of the subject.

4. The method according to claim 3, wherein the cholinergic neurodegenerative disease is selected from the group consisting of Alzheimer's disease, dysmnesia, learning disability, schizophrenia, cognitive dysfunction, hyperactivity disorder, anxiety neurosis, depression, analgesia and Parkinson's disease.

5. A method for preparing the compound of formula (I) according to claim 1, comprising (a) preparing a reaction mixture by adding a compound of following formula (III): 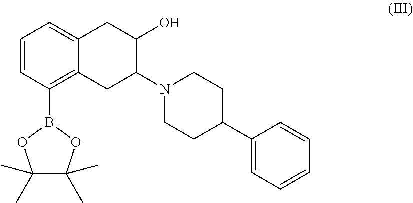 [SMILES: CC1(C)OB(c2cccc3c2CC(N2CCC(c4ccccc4)CC2)C(O)C3)OC1(C)C]
to reactive fluorine ions ¹⁸F,
followed by (b) performing a chiral separation of said reaction mixture obtained after step (a).

6. The method according to claim 5, wherein step (b) is performed by loading the reaction mixture obtained after step (a) onto a semi-preparative chiral column using a chiral mobile phase comprising a mixture of acetonitrile, ammonium acetate and methanol.

7. The method according to claim 6, wherein the mobile phase comprises from 50% to 90% by volume of acetonitrile, from 0% to 20% by volume of ammonium acetate and from 0% to 40% by volume of methanol, relative to the total volume of said mobile phase.

8. The method according to claim 5, wherein the compound of formula (III) is obtained with a method comprising:
performing a diazotization reaction of a compound of following compound (IV): 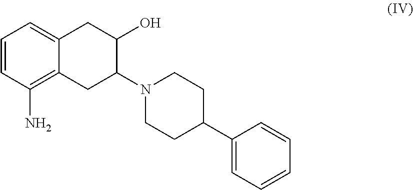 [SMILES: Nc1cccc2c1CC(N1CCC(c3ccccc3)CC1)C(O)C2]
then
performing a substitution reaction with a halogen on the diazo compound obtained by the diazotization reaction,
said step comprising reacting the compound of formula (IV) with sodium nitrite, followed by the addition of potassium iodide,
to obtain a compound of following formula (V): 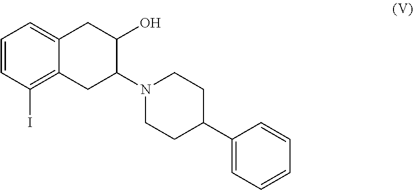 [SMILES: OC1Cc2cccc(I)c2CC1N1CCC(c2ccccc2)CC1]
and
converting the compound of formula (V) by Miyaura borylation to obtain a compound of formula (III).

9. A compound having the following formula (II):

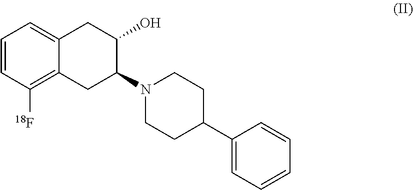 [SMILES: O[C@H]1Cc2cccc([18F])c2C[C@@H]1N1CCC(c2ccccc2)CC1]

10. A compound having the following formula (III): 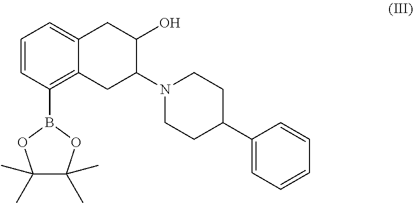 [SMILES: CC1(C)OB(c2cccc3c2CC(N2CCC(c4ccccc4)CC2)C(O)C3)OC1(C)C]
